(19) United States
(12) Patent Application Publication
(10) Pub. No.: US 2009/0326658 A1
(43) Pub. Date: Dec. 31, 2009

(54) INTERVERTEBRAL PROSTHETIC DISC AND METHOD OF INSTALLING SAME

(75) Inventor: Randall N. Allard, Germantown, TN (US)

Correspondence Address: MEDTRONIC;Attn: Noreen Johnson - IP Legal Department, 2600 Sofamor Danek Drive, MEMPHIS, TN, 38132, US

(73) Assignee: SDGI HOLDINGS, INC., Wilmington, DE (US)

(21) Appl. No.: 12/512,957

(22) Filed: Jul. 30, 2009

Related U.S. Application Data
(62) Division of application No. 11/339,308, filed on Jan. 25, 2006, now abandoned.

(51) Int. Cl. A61F 2/44

(52) U.S. Cl. 623/17.16

(57) ABSTRACT

An intervertebral prosthetic disc that is configured to be installed within an intervertebral space that can be established between an inferior vertebra and a superior vertebra is disclosed. The intervertebral prosthetic disc includes an inferior articular half that can be configured to engage the inferior vertebra and a superior articular half that can be configured to engage the superior vertebra. The inferior articular half can be configured to cooperate with the superior articular half to allow relative angular motion between the inferior vertebra and the superior vertebra when installed. Further, the intervertebral prosthetic device can be sized and shaped to pass through a psoas muscle without injuring a spinal cord or a sympathetic chain.

### FIELD OF THE DISCLOSURE

The present disclosure relates generally to orthopedics and spinal surgery. More specifically, the present disclosure relates to intervertebral prosthetic discs.

### BACKGROUND

In human anatomy, the spine is a generally flexible column that can take tensile and compressive loads. The spine also allows bending motion and provides a place of attachment for ribs, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of “wear and tear”.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is an anterior view of a portion of a vertebral column;

FIG. 3 is a lateral view of a pair of adjacent vertrebrae;

FIG. 4 is a top plan view of a vertebra;

FIG. 5 is a posterior view of a first embodiment of an intervertebral prosthetic disc;

FIG. 6 is an exploded posterior view of the first embodiment of the intervertebral prosthetic disc;

FIG. 7 is a lateral view of the first embodiment of the intervertebral prosthetic disc;

FIG. 8 is an exploded lateral view of the first embodiment of the intervertebral prosthetic disc;

FIG. 9 is a plan view of an inferior half of the first embodiment of the intervertebral prosthetic disc;

FIG. 10 is another plan view of the inferior half of the first embodiment of the intervertebral prosthetic disc;

FIG. 11 is a plan view of a superior half of the first embodiment of the intervertebral prosthetic disc;

FIG. 12 is an exploded lateral view of the first embodiment of the intervertebral prosthetic disc installed within an intervertebral space between a pair of adjacent vertrebrae;

FIG. 13 is an anterior view of the first embodiment of the intervertebral prosthetic disc installed within an intervertebral space between a pair of adjacent vertrebrae;

FIG. 14 is a lateral view of the first embodiment of the intervertebral prosthetic disc installed within an intervertebral space between a pair of adjacent vertrebrae;

FIG. 15 is a plan view of the inferior half of the first embodiment of the intervertebral prosthetic disc disposed over a vertebral body;

FIG. 16 is a flow chart of a method of installing an intervertebral prosthetic disc within an intervertebral space between a pair of adjacent vertebrae;

FIG. 17 is a posterior view of a second embodiment of an intervertebral prosthetic disc;

FIG. 18 is an exploded posterior view of the second embodiment of the intervertebral prosthetic disc;

FIG. 19 is a lateral view of the second embodiment of the intervertebral prosthetic disc;

FIG. 20 is an exploded lateral view of the second embodiment of the intervertebral prosthetic disc;

FIG. 21 is a plan view of an inferior half of the second embodiment of the intervertebral prosthetic disc;

FIG. 22 is another plan view of the inferior half of the first embodiment of the intervertebral prosthetic disc;

FIG. 23 is a posterior view of a third embodiment of an intervertebral prosthetic disc;

FIG. 24 is an exploded posterior view of the third embodiment of the intervertebral prosthetic disc;

FIG. 25 is a lateral view of the third embodiment of the intervertebral prosthetic disc;

FIG. 26 is an exploded lateral view of the third embodiment of the intervertebral prosthetic disc;

FIG. 27 is a plan view of an inferior half of the third embodiment of the intervertebral prosthetic disc;

FIG. 28 is a posterior view of a fourth embodiment of an intervertebral prosthetic disc;

FIG. 29 is an exploded posterior view of the fourth embodiment of the intervertebral prosthetic disc;

FIG. 30 is a lateral view of the fourth embodiment of the intervertebral prosthetic disc;

FIG. 31 is an exploded lateral view of the fourth embodiment of the intervertebral prosthetic disc;

FIG. 32 is a plan view of an inferior half of the fourth embodiment of the intervertebral prosthetic disc;

FIG. 33 is another plan view of the inferior half of the fourth embodiment of the intervertebral prosthetic disc;

FIG. 34 is a plan view of a superior half of the fourth embodiment of the intervertebral prosthetic disc;

FIG. 35 is plan view of another embodiment of an inferior half of an intervertebral prosthetic disc; and

FIG. 36 is a plan view of yet another embodiment of an inferior half of an intervertebral prosthetic disc.

### DETAILED DESCRIPTION OF THE DRAWINGS

An intervertebral prosthetic disc that is configured to be installed within an intervertebral space that can be established between an inferior vertebra and a superior vertebra is disclosed. The intervertebral prosthetic disc includes an inferior articular half that can be configured to engage the inferior vertebra and a superior articular half that can be configured to engage the superior vertebra. The inferior articular half can be configured to cooperate with the superior articular half to allow relative angular motion between the inferior vertebra and the superior vertebra when installed. Further, the intervertebral prosthetic device can be sized and shaped to pass through a psoas muscle without injuring a spinal cord or a sympathetic chain.

In another embodiment, an intervertebral prosthetic disc that is configured to be installed within an intervertebral space that can be established between an inferior vertebra and a superior vertebra is disclosed. The intervertebral prosthetic disc includes an inferior articular half that can include an inferior articular surface, a projection that can extend from the inferior articular surface, an inferior bearing surface, and at least one inferior rib that can extend from the inferior bearing surface. The inferior rib can be configured to engage a cortical rim of the inferior vertebra. Also, the inferior rib can have a height that is less than or equal to six millimeters (6 mm).

In yet another embodiment, a method of installing an intervertebral prosthetic disc within an intervertebral space that can be established between an inferior vertebra and a superior vertebra of a patient is disclosed. The method includes laterally inserting an insertion device into the patient. The insertion device can be configured to deliver a fusion device or the intervertebral prosthetic disc to the intervertebral space. Moreover, the method includes delivering the intervertebral prosthetic disc to the intervertebral space with the insertion device.

### Description of Relevant Anatomy

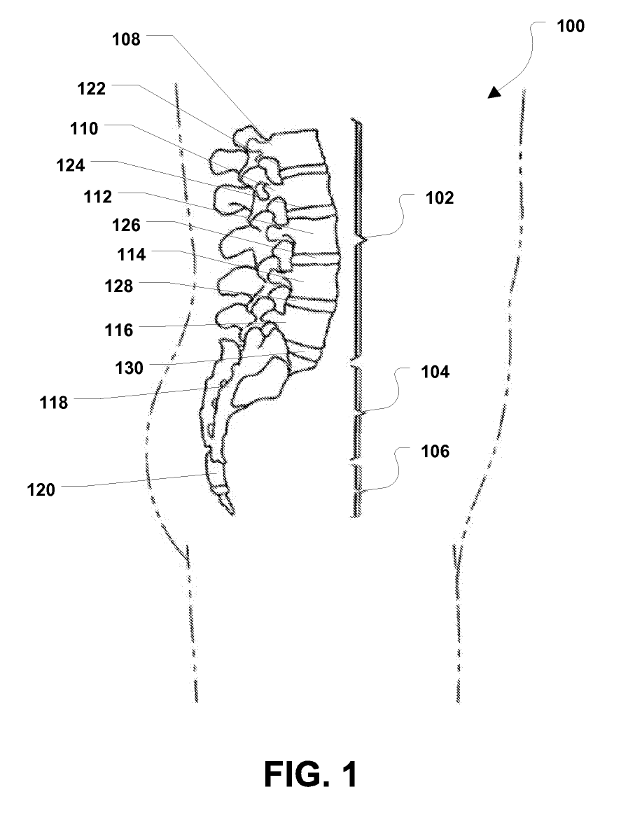

Referring initially to FIG. 1, a portion of a vertebral column, designated 100, is shown. As depicted, the vertebral column 100 includes a lumber region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumber vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumber vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

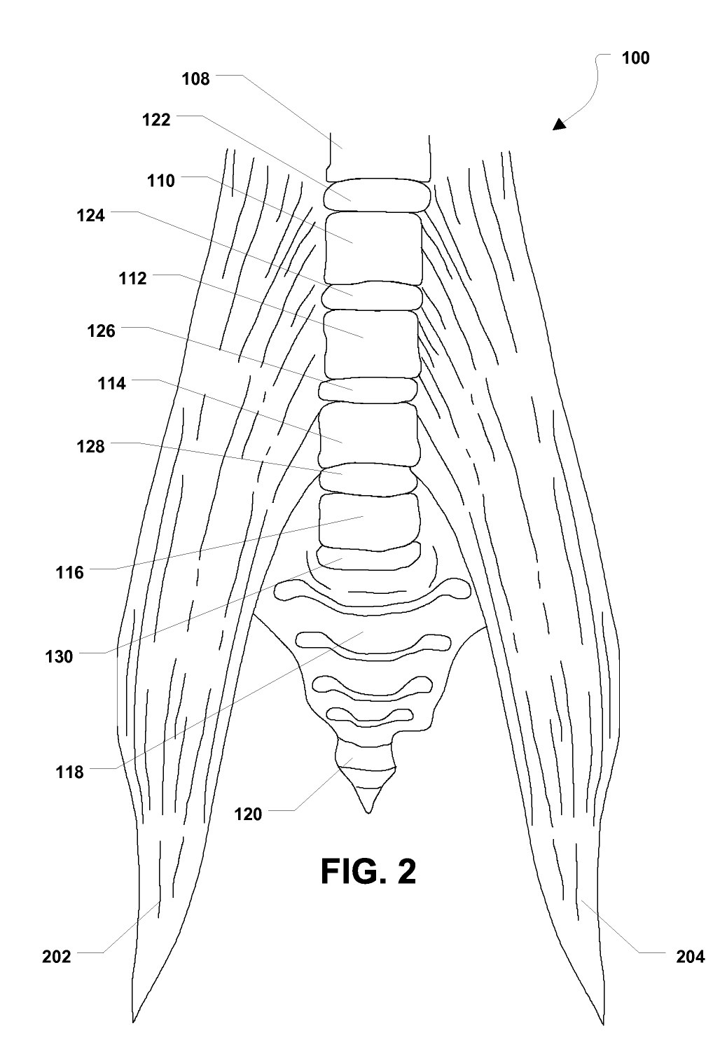

FIG. 2 illustrates the psoas muscles 202, 204 that can extend from the vertebral column 100. For clarity, only the psoas major muscles are shown in FIG. 2. In a particular embodiment, an intervertebral prosthetic disc according to one or more of the embodiments described herein can be implanted through one of the psoas muscles 202, 204 using a lateral surgical approach, described in detail below.

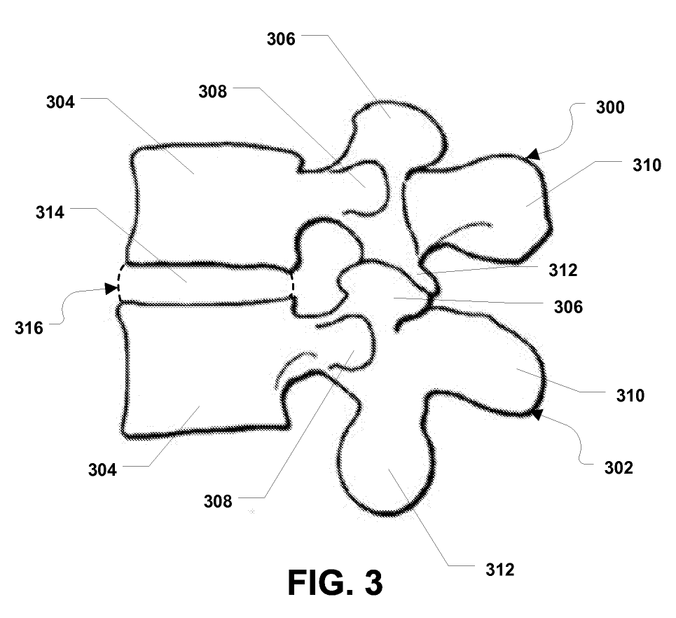

FIG. 3 depicts a detailed lateral view of two adjacent vertebrae, e.g., two of the lumbar vertebra 108, 110, 112, 114, 116 shown in FIG. 1 and FIG. 2. FIG. 3 illustrates a superior vertebra 300 and an inferior vertebra 302. As shown, each vertebra 300, 302 includes a vertebral body 304, a superior articular process 306, a transverse process 308, a spinous process 310 and an inferior articular process 312. FIG. 3 further depicts an intervertebral space 314 that can be established between the superior vertebra 300 and the inferior vertebra 302 by removing an intervertebral disc 316 (shown in dashed lines). As described in greater detail below, an intervertebral prosthetic disc according to one or more of the embodiments described herein can be installed within the intervertebral space 312 between the superior vertebra 300 and the inferior vertebra 302.

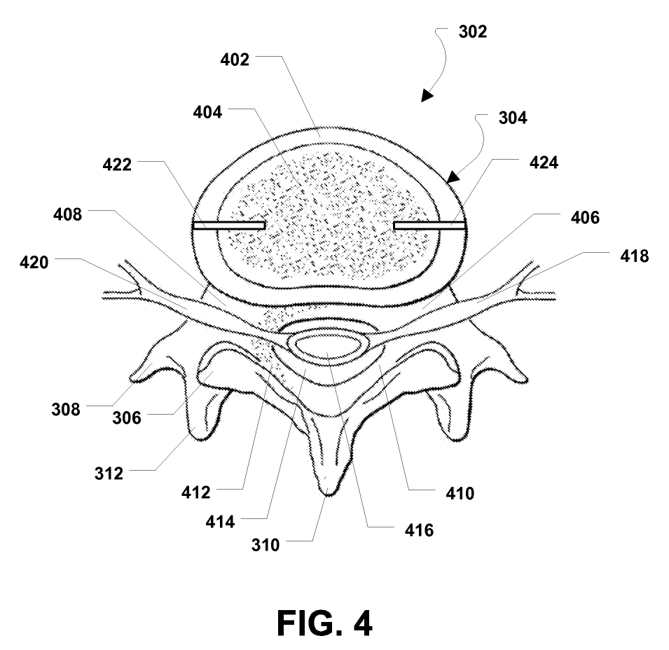

Referring to FIG. 4, a vertebra, e.g., the inferior vertebra 302 (FIG. 3), is illustrated. As shown, the vertebral body 304 of the inferior vertebra 302 includes a cortical rim 402 composed of cortical bone. Also, the vertebral body 304 includes cancellous bone 404 within the cortical rim 402. The cortical rim 402 is often referred to as the apophyseal rim or apophyseal ring. Further, the cancellous bone 404 is softer and weaker than the cortical bone of the cortical rim 402.

As illustrated in FIG. 4, the inferior vertebra 302 further includes a first pedicle 406, a second pedicle 408, a first lamina 410, and a second lamina 412. Further, a vertebral foramen 414 is established within the inferior vertebra 302. A spinal cord 416 passes through the vertebral foramen 414. Moreover, a first nerve root 418 and a second nerve root 420 extend from the spinal cord 416.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 3 and FIG. 4. The first and second cervical vertebrae are structurally different than the rest of the vertebrae in order to support a skull.

FIG. 4 further depicts a first slot 422 and a second slot 424 that can be established within the cortical rim 402 of the inferior vertebra 302. In a particular embodiment, the first slot 422 and the second slot 424 are established during surgery to install an intervertebral prosthetic disc according to one or more of the embodiments described herein. The first slot 422 and the second slot 424 can be established using a cutting device, e.g., a chisel that is designed to cut a groove, or slot, in a vertebra, prior to the installation of the intervertebral prosthetic disc. Further, the first slot 422 and the second slot 424 are sized and shaped to receive and engage a first rib and a second rib, described in detail below, that extend from an intervertebral prosthetic disc according to one or more of the embodiments described herein. The first slot 422 and the second slot 424 can cooperate with a first rib and second rib to facilitate proper alignment of an intervertebral prosthetic disc within an intervertebral space between an inferior vertebra and a superior vertebra.

### Description of a First Embodiment

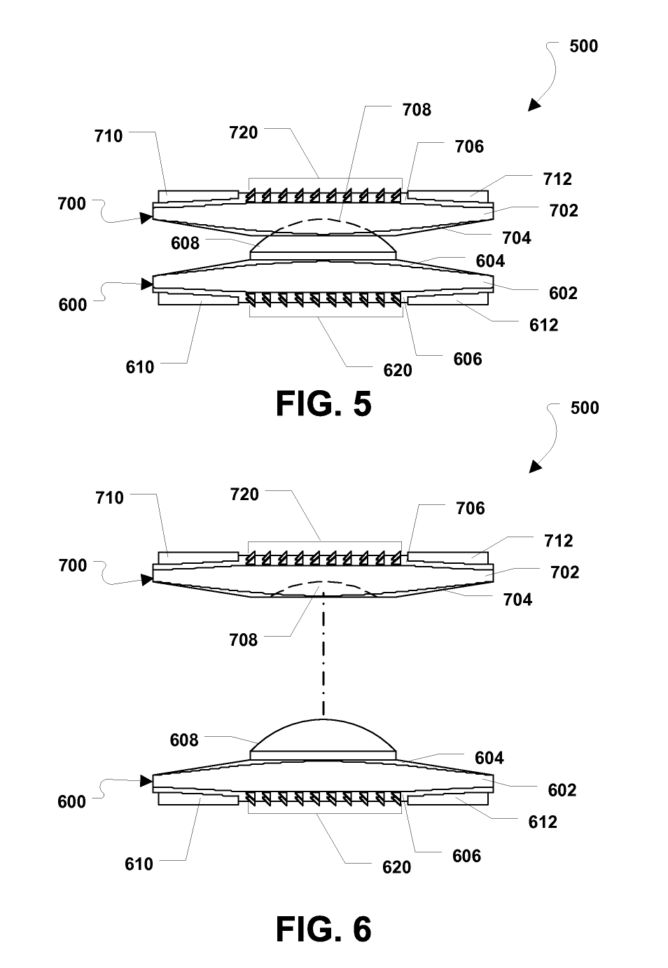

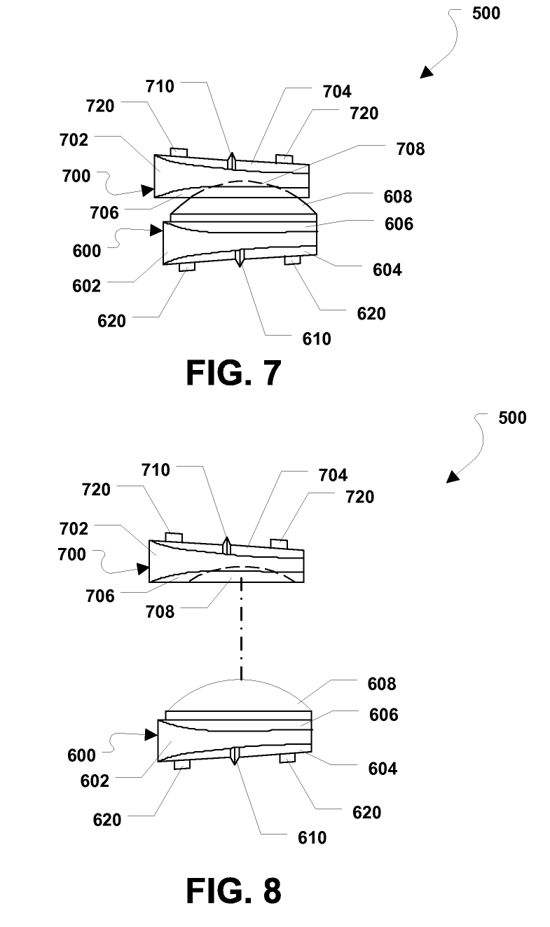

Referring to FIGS. 5 through 11 a first embodiment of an intervertebral prosthetic disc is shown and is generally designated 500. As illustrated, the intervertebral prosthetic disc 500 includes an inferior articular half 600 and a superior articular half 700. In a particular embodiment, the articular halves 600, 700 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the articular halves 600, 700 can be made from any other substantially rigid biocompatible materials.

In a particular embodiment, the inferior articular half 600 includes an inferior support plate 602 that has an inferior articular surface 604 and an inferior bearing surface 606. In a particular embodiment, the inferior articular surface 604 and the inferior bearing surface 606 are generally rounded. In a particular embodiment, after installation the inferior bearing surface 606 can be in direct contact with vertebral bone, e.g., cortical bone and cancellous bone. Further, the inferior bearing surface 606 can be coated with a bone-growth promoting substance, e.g., a hydroxyapatite coating formed of calcium phosphate. Additionally, the inferior bearing surface 606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 5 through FIG. 11, a projection 608 extends from the inferior articular surface 604 of the inferior support plate 602. In a particular embodiment, the projection 608 has a hemi-spherical shape. Alternatively, the projection 608 can have an elliptical shape, a cylindrical shape, or other arcuate shape.

As further illustrated in FIG. 5 through 11, the inferior articular half 600 includes a first inferior rib 610 and a second inferior rib 612 that extend substantially perpendicularly from the inferior bearing surface 606. In a particular embodiment, as shown in FIG. 10, the first inferior rib 610 and the second inferior rib 612 extend along a longitudinal axis 614 defined by the inferior articular half 600. As shown, the first inferior rib 610 and the second inferior rib 612 can extend along the longitudinal axis 614 from a perimeter of the inferior articular half 600 toward a lateral axis 616 that is defined by the inferior articular half 600. In a particular embodiment, the first inferior rib 610 and the second inferior rib 612 are sized and shaped to engage a first and second slot, e.g., the first slot 422 and the second slot 424 within the cortical rim 402 of the inferior vertebra 302 shown in FIG. 4.

FIG. 5 through FIG. 11 also show that the inferior articular half 600 includes a plurality of inferior teeth 620 that extend from the inferior bearing surface 606. As shown, in a particular embodiment, the inferior teeth 620 are generally saw-tooth, or triangle, shaped. Further, the inferior teeth 620 are designed to engage cancellous bone, e.g., the cancellous bone 404 of the inferior vertebra 302 shown in FIG. 4. Additionally, the inferior teeth 620 can prevent the inferior articular half 600 from moving with respect to the inferior vertebra 302 after the intervertebral prosthetic disc 500 is installed within the intervertebral space 314 (FIG. 3) between the inferior vertebra 302 (FIG. 3) and the superior vertebra 300 (FIG. 3).

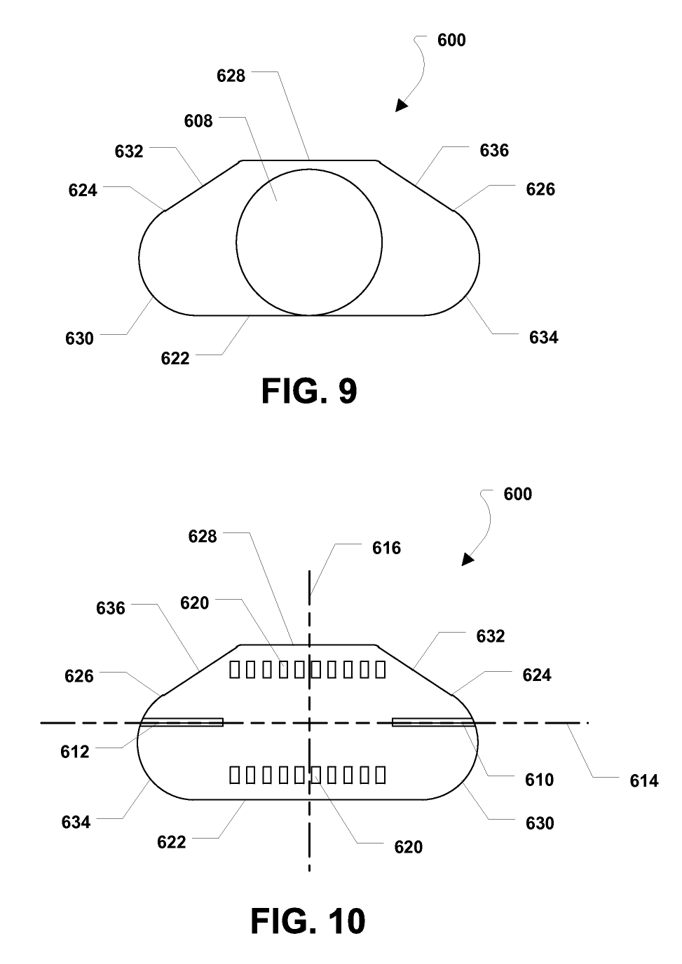

As illustrated in FIG. 9 and FIG. 10, the inferior articular half 600 can be generally shaped to match the general shape of the vertebral body of a vertebra, e.g., the vertebral body 304 of the inferior vertebra 302 shown in FIG. 3. For example, the inferior articular half 600 can have a general trapezoid shape and the inferior articular half 600 can include a posterior side 622. A first lateral side 624 and a second lateral side 626 can extend from the posterior side 622 to an anterior side 628. In a particular embodiment, the first lateral side 624 includes a curved portion 630 and a straight portion 632 that extends at an angle toward the anterior side 628. Further, the second lateral side 626 can also include a curved portion 634 and a straight portion 636 that extends at an angle toward the anterior side 628.

As shown in FIG. 9 and FIG. 10, the anterior side 628 of the inferior articular half 600 can be relatively shorter than the posterior side 622 of the inferior articular half 600. Further, in a particular embodiment, the anterior side 628 is substantially parallel to the posterior side 622. As indicated in FIG. 9, the projection 608 can be situated, or otherwise formed, on the inferior articular surface 604 such that the perimeter of the projection 608 is tangential to the posterior side 622 of the inferior articular half 600. In alternative embodiments (not shown), the projection 608 can be situated, or otherwise formed, on the inferior articular surface 604 such that the perimeter of the projection 608 is tangential to the anterior side 628 of the inferior articular half 600 or tangential to both the anterior side 628 and the posterior side 622. In a particular embodiment, the projection 608 and the inferior support plate 602 comprise a monolithic body.

In a particular embodiment, the superior articular half 700 includes a superior support plate 702 that has a superior articular surface 704 and a superior bearing surface 706. In a particular embodiment, the superior articular surface 704 and the superior bearing surface 706 are generally rounded. In a particular embodiment, after installation the superior bearing surface 706 can be in direct contact with vertebral bone, e.g., cortical bone and cancellous bone. Further, the superior bearing surface 706 can be coated with a bone-growth promoting substance, e.g., a hydroxyapatite coating formed of calcium phosphate. Additionally, the superior bearing surface 706 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 5 through FIG. 11, a depression 708 extends into the superior articular surface 704 of the superior support plate 702. In a particular embodiment, the depression 708 is sized and shaped to receive the projection 608 of the inferior articular half 600. For example, the depression 708 can have a hemi-spherical shape. Alternatively, the depression 708 can have an elliptical shape, a cylindrical shape, or other arcuate shape.

As further illustrated in FIG. 5 through 11, the superior articular half 700 includes a first superior rib 710 and a second superior rib 712 that extend substantially perpendicularly from the superior bearing surface 706. In a particular embodiment, the first superior rib 710 and the second superior rib 712 of the superior articular half 700 are arranged in a manner similar to the first inferior rib 610 and the second inferior rib 612 of the inferior articular half 600, as shown in FIG. 10. In another particular embodiment, the first superior rib 710 and the second superior rib 712 are sized and shaped to engage a first and second slot, e.g., the first slot 422 and the second slot 424 within the cortical rim 402 of the superior vertebra 302 shown in FIG. 4.

FIG. 5 through FIG. 11 also show that the superior articular half 700 includes a plurality of superior teeth 720 that extend from the superior bearing surface 706. As shown, in a particular embodiment, the superior teeth 720 are generally saw-tooth, or triangle, shaped. Further, the superior teeth 720 are designed to engage cancellous bone, e.g., the cancellous bone 404 of the superior vertebra 302 shown in FIG. 4. Additionally, the superior teeth 720 can prevent the superior articular half 700 from moving with respect to the superior vertebra 302 after the intervertebral prosthetic disc 500 is installed within the intervertebral space 314 (FIG. 3) between the inferior vertebra 302 (FIG. 3) and the superior vertebra 300 (FIG. 3).

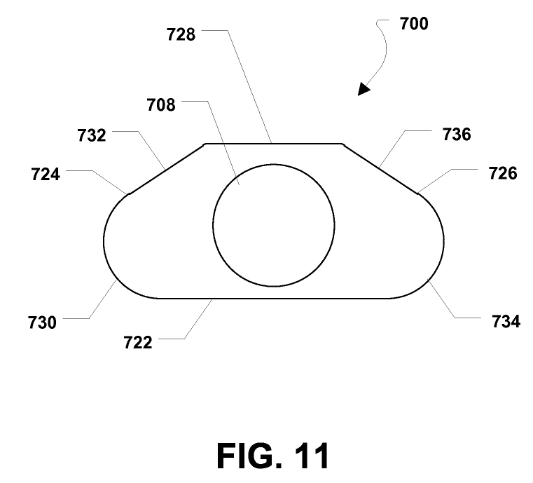

In a particular embodiment, the superior articular half 700 can be shaped to match the shape of the inferior articular half 600, shown in FIG. 9 and FIG. 10. Further, the superior articular half 700 can be shaped to match the general shape of the vertebral body of a vertebra, e.g., the vertebral body 304 of the superior vertebra 302 shown in FIG. 3. For example, as shown in FIG. 11, the superior articular half 700 can have a general trapezoid shape and the superior articular half 700 can include a posterior side 722. A first lateral side 724 and a second lateral side 726 can extend from the posterior side 722 to an anterior side 728. In a particular embodiment, the first lateral side 724 includes a curved portion 730 and a straight portion 732 that extends at an angle toward the anterior side 728. Further, the second lateral side 726 can also include a curved portion 734 and a straight portion 736 that extends at an angle toward the anterior side 728.

As shown in FIG. 9 and FIG. 10, the anterior side 728 of the superior articular half 700 can be relatively shorter than the posterior side 722 of the superior articular half 700. Further, in a particular embodiment, the anterior side 728 is substantially parallel to the posterior side 722.

In a particular embodiment, the overall height of the intervertebral prosthetic device 500 can be in a range from six millimeters to twenty-two millimeters (6-22 mm). Further, the installed height of the intervertebral prosthetic device 500 can be in a range from four millimeters to sixteen millimeters (4-16 mm). In a particular embodiment, the installed height can be substantially equivalent to the distance between an inferior vertebra and a superior vertebra when the intervertebral prosthetic device 500 is installed there between.

In a particular embodiment, the length of the intervertebral prosthetic device 500, e.g., along a longitudinal axis, can be in a range from thirty-three millimeters to fifty millimeters (33-50 mm). Additionally, the width of the intervertebral prosthetic device 500, e.g., along a lateral axis, can be in a range from eighteen millimeters to twenty-nine millimeters (18-29 mm). Moreover, in a particular embodiment, each rib 610, 612, 710, 712 can have a height in a range from one millimeter to six millimeters (1-6 mm). In a particular embodiment, the height of each rib 610, 612, 710, 712 is measured at a location of each rib 610, 612, 710, 712 nearest to the center of each half 600, 700 of the intervertebral prosthetic device 500.

In a particular embodiment, the ribs 610, 612, 710, 712 can be considered “low profile”. Further, intervertebral prosthetic disc 500 can be considered to be “low profile.” The low profile of the ribs 610, 612, 710, 712 and the intervertebral prosthetic device 500 can allow the intervertebral prosthetic device 500 to be implanted into an intervertebral space between an inferior vertebra and a superior vertebra laterally through a patient's psoas muscle, e.g., through an insertion device. Accordingly, the risk of damage to a patient's spinal cord or sympathetic chain can be substantially minimized. In alternative embodiments, all of the superior and inferior teeth 620, 720 can be oriented to engage in a direction substantially opposite the direction of insertion of the prosthetic disc into the intervertebral space.

Further, the intervertebral prosthetic disc 500 can have a general “bullet” shape as shown in the posterior plan view, described herein. The bullet shape of the intervertebral prosthetic disc 500 provided by the rounded bearing surfaces 604, 704 can further allow the intervertebral prosthetic disc 500 to be inserted through the patient's psoas muscle while minimizing risk to the patient's spinal cord and sympathetic chain.

### Installation of the First Embodiment within an Intervertebral Space

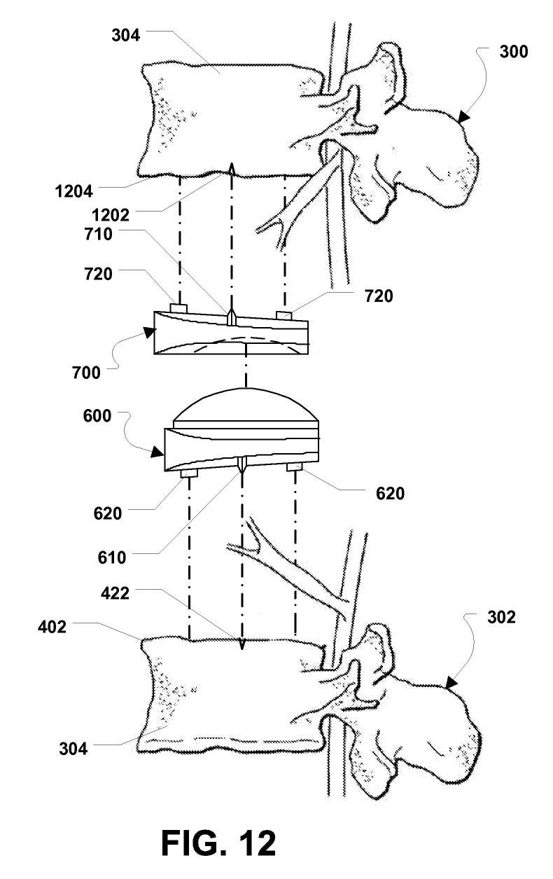

Referring to FIG. 12 through FIG. 14, an intervertebral prosthetic disc is shown between the superior vertebra 300 and the inferior vertebra 302, previously introduced and described in conjunction with FIG. 3. In a particular embodiment, the intervertebral prosthetic disc is the intervertebral prosthetic disc 500 described in conjunction with FIG. 5 through FIG. 11. Alternatively, the intervertebral prosthetic disc can be an intervertebral prosthetic disc according to any of the embodiments disclosed herein.

As shown in FIG. 12 through FIG. 14, the intervertebral prosthetic disc 500 is installed within the intervertebral space 314 that can be established between the superior vertebra 300 and the inferior vertebra 302 by removing vertebral disc material (not shown). FIG. 12 shows that the inferior teeth 620 of the inferior articular half 600 can engage the cancellous bone of the inferior vertebra 302. Further, the first inferior rib 610 of the inferior articular half 600 can engage a first slot 422 that can be established within the vertebral body 304 of the inferior vertebra 302. In particular, the first slot 422 can be established within the cortical rim 402 of the vertebral body 304 of the inferior vertebra 302. A second inferior rib (not shown in FIG. 12) of the inferior articular half 600 can engage a second slot (not shown in FIG. 12) that can be established within the vertebral body 304 of the inferior vertebra 302.

FIG. 12 also indicates that the superior teeth 720 of the superior articular half 700 can engage the cancellous bone of the superior vertebra 300. Moreover, the first superior rib 710 of the superior articular half 700 can engage a first slot 1202 that is established within the vertebral body 304 of the superior vertebra 300. In particular, the first slot 1200 can be established within the cortical rim 1204 of the vertebral body 304 of the superior vertebra 300. A second superior rib (not shown in FIG. 12) of the superior articular half 700 can engage a second slot (not shown in FIG. 12) that can be established within the vertebral body 304 of the superior vertebra 302.

As illustrated in FIG. 12 through FIG. 14, the projection 608 that extends from the inferior articular half 600 of the intervertebral prosthetic disc 500 can engage the depression 708 that is formed within the superior articular half 700 of the intervertebral prosthetic disc 500. It is to be appreciated that when the intervertebral prosthetic disc 500 is installed between the superior vertebra 300 and the inferior vertebra 302, the intervertebral prosthetic disc 500 allows relative motion between the superior vertebra 300 and the inferior vertebra 302. Specifically, the configuration of the inferior articular half 600 and the superior articular half 700 allows the inferior articular half 600 to rotate with respect to the superior articular half 700. As such, the superior vertebra 300 can rotate with respect to the inferior vertebra 302.

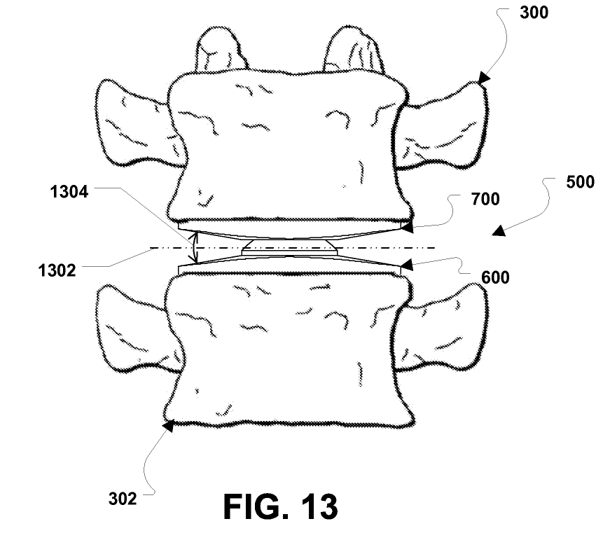

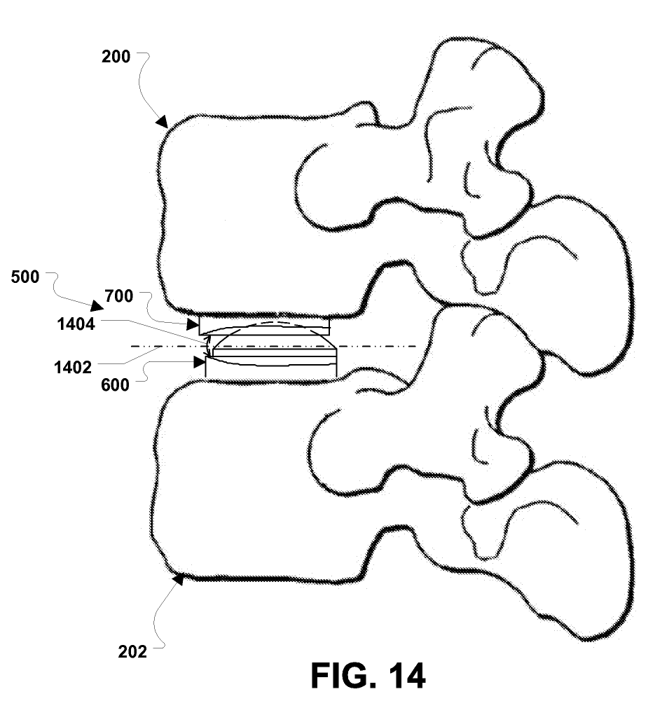

In a particular embodiment, the intervertebral prosthetic disc 500 can allow angular movement in any radial direction relative to the intervertebral prosthetic disc 500. For example, FIG. 13 indicates that the inferior articular half 600 and the superior articular half 700 can move relative to each other through a longitudinal axis 1302 over an angle 1304. In a particular embodiment, the angle 1304 of movement through the longitudinal axis 1302 is plus or minus twelve degrees (±12°) relative to the longitudinal axis 1302. Additionally, FIG. 14 indicates that the inferior articular half 600 and the superior articular half 700 can move relative to each other through a lateral axis 1402 over an angle 1404. In a particular embodiment, the angle 1404 of movement through the lateral axis 1402 is plus or minus sixteen degrees (±16°) relative to the lateral axis 1402.

Further, as depicted in FIG. 12 through 14, the inferior articular half 600 can be placed on the inferior vertebra 302 so that the center of rotation of the inferior articular half 600 is substantially aligned with the center of rotation of the inferior vertebra 302. Similarly, the superior articular half 700 can be placed relative to the superior vertebra 300 so that the center of rotation of the superior articular half 700 is substantially aligned with the center of rotation of the superior vertebra 300. Accordingly, when the vertebral disc, between the inferior vertebra 302 and the superior vertebra 300, is removed and replaced with the intervertebral prosthetic disc 500 the relative motion of the vertebrae 300, 302 provided by the vertebral disc is substantially replicated.

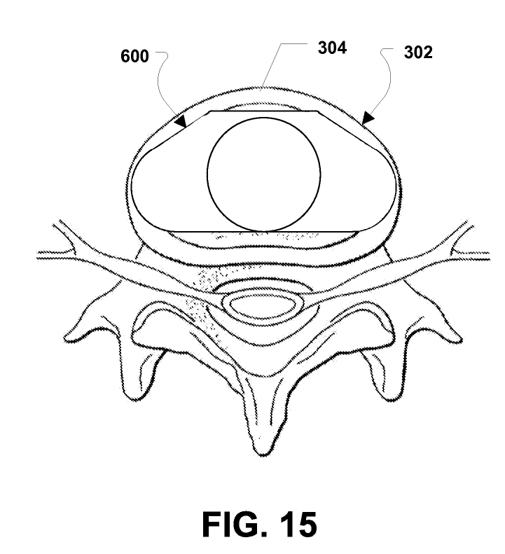

Referring to FIG. 15, the inferior articular half 600 is shown disposed on top of the inferior vertebra 302. FIG. 15 shows that the shape of the inferior articular half 600 generally resembles the shape of the vertebral body 304, i.e., the shape of the vertebral body 304 from a top plan view. In a particular embodiment, the shape of the superior articular half 700 (not shown in FIG. 15) is similar to the shape of the inferior articular half 600 and also generally resembles the shape of the vertebral body 304 in the plan view.

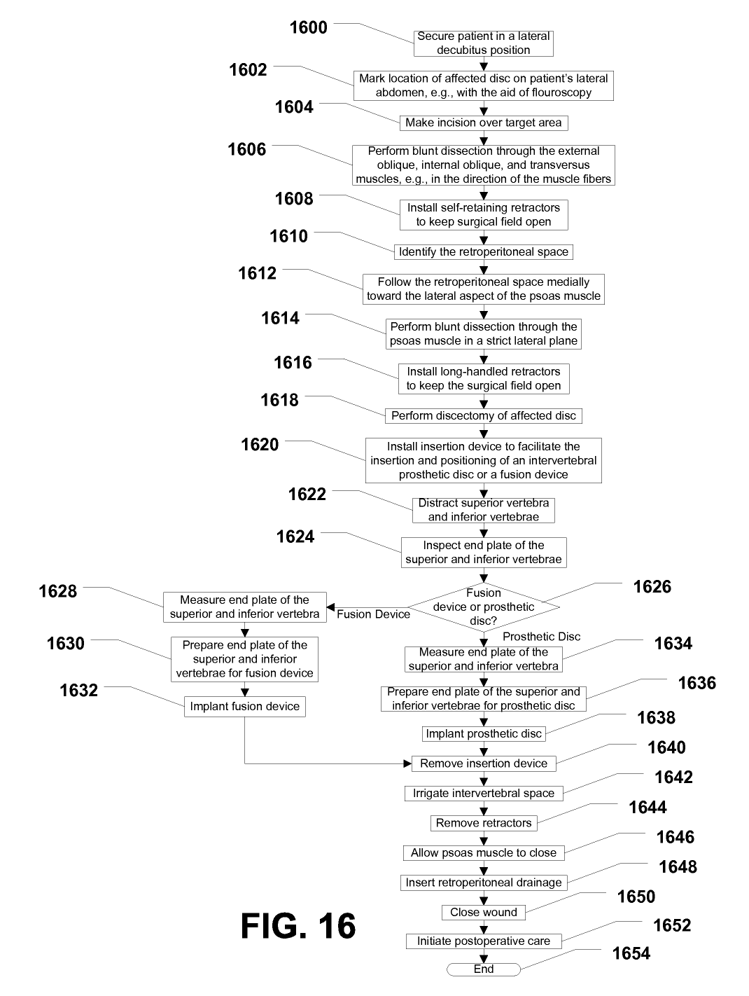

FIG. 16 depicts a method of installing an intervertebral prosthetic disc between a superior vertebra and an inferior vertebra. Commencing at block 1600, the patient is secured in such a way to provide a lateral approach to a damaged or diseased vertebral disc. For example, the patient can be secured in a lateral decubitus position. Further, the patient can be secured in a lateral decubitus position on an adjustable surgical table. In a particular embodiment, the surgical table can be flexed slightly in order to arch the patient's side. Arching the patient's side can increase the distance between the iliac crest and the inferior border of the twelfth rib.

Moving to block 1602, the location of the affected disc is marked on patient's lateral abdomen, e.g., with the aid of fluoroscopy. At block 1604, an incision is made over the target area. At block 1606, blunt dissection can be performed through the external oblique, internal oblique, and transversus muscles, e.g., in the direction of the muscle fibers. Further, at block 1608, self-retaining retractors can be installed to keep surgical field open.

Continuing to block 1610, the retroperitoneal space can be identified. At block 1612, the retroperitoneal space can be followed medially toward the lateral aspect of the psoas muscle. Thereafter, at block 1614, blunt dissection can be performed through the psoas muscle in a strict lateral plane. In a particular embodiment, if the psoas muscle is accessed correctly and a strict lateral path is taken through the muscle, a tunnel can be formed through the psoas muscle and the fibers of the muscle can protect the sympathetic nerve chain and the nerve roots that exit the spine. In a particular embodiment, the tunnel is an anatomic safe zone that is approximately two to three centimeters wide through the psoas muscle. Further, the intervertebral prosthetic device according to one or more of the embodiments disclosed herein is designed to pass through the anatomic safe zone without causing injury or damage to the spinal cord or the sympathetic chain.

At block 1616, long-handled retractors can be installed to keep the surgical field open. Proceeding to block 1618, a discectomy of affected disc is performed to remove the affected disc. At block 1620, an insertion device can be installed. In a particular embodiment, the insertion device can facilitate the insertion and positioning of an intervertebral prosthetic disc or a fusion device to replace the affected disc that is removed. Moving to block 1622, the superior vertebra and inferior vertebra are distracted to increase the intervertebral space between the superior vertebra and the inferior vertebra. At block 1624, the end place of the superior vertebra and the end plate of the inferior vertebra are inspected in order to determine the damage that may be caused by the affected disc. Such damage can include bone degeneration of either end plate.

Continuing to decision step 1626, it is determined whether to install a fusion device or a prosthetic disc. In a particular embodiment, the prosthetic disc is one of the intervertebral prosthetic discs described herein. Further, the determination to install a fusion device or a prosthetic device can be based, at least in part, on the level of damage to the end plates of the superior vertebra and the inferior vertebra. Also, the determination to install a fusion device or a prosthetic device can be based on the stability of the motion segment that includes the superior vertebra and the inferior vertebra and the surgical access. If the decision is made to install a fusion device, the method proceeds to block 1628 and the end plate of the superior vertebra and the end plate of the inferior vertebra are measured to determine what size fusion device is needed for implantation.

At block 1630, the end plate of the superior vertebra and the end plate of the inferior vertebra are prepared to receive the fusion device. In a particular embodiment, this preparation may include removing portions of the cortical rim of each vertebra. Further, this preparation may include cutting one or more slots in the cortical rim of each vertebra. Moving to block 1632, the fusion device can be implanted, or otherwise disposed, within the intervertebral space that is established between the superior vertebra and the inferior vertebra.

Returning to decision step 1626, when the decision is made to implant an intervertebral prosthetic disc, the method moves to block 1634 and the end plate of the superior vertebra and the end plate of the inferior vertebra are measured to determine what size intervertebral prosthetic disc is needed for implantation. At block 1636, the end plate of the superior vertebra and the end plate of the inferior vertebra are prepared to receive the intervertebral prosthetic disc. In a particular embodiment, this preparation may include removing portions of the cortical rim of each vertebra. Further, this preparation may include cutting one or more slots in the cortical rim of each vertebra. In a particular embodiment, one or more of the same tools can be used to prepare the end plates when installing an intervertebral prosthetic disc and when installing a fusion device. Moving to block 1638, the intervertebral prosthetic disc can be implanted, or otherwise disposed, within the intervertebral space that is established between the superior vertebra and the inferior vertebra.

After the fusion device is implanted at block 1632 or the intervertebral prosthetic disc is implanted at block 1638, the method proceeds to block 1640 and the insertion device is removed. At block 1642, the intervertebral space is irrigated. Further, at block 1644, the retractors are removed. Moving to block 1646, the psoas muscle can be allowed to close. At block 1648, a retroperitoneal drainage can be inserted into the wound. Additionally, at block 1650, the wound can be closed. In a particular embodiment, the wound can be closed by applying adaptation sutures to the three muscle layers in the abdominal wall and by repairing the subcutaneous tissue in such a way to properly align the overlying dermis. Thereafter, subcuticular closure of the skin can be performed and sterile connective strips can be applied across the closed incision to facilitate healing and reduce scarring. Moving to block 1652, postoperative care can be initiated. The method ends at step 1654.

In a particular embodiment, an inserter tool can be used facilitate implanting the intervertebral prosthetic disc according to one or more of the embodiments described herein. The inserter tool can engage the intervertebral prosthetic disc and hold the intervertebral prosthetic disc in a flexed position so that an insertion height of a leading edge of the intervertebral prosthetic disc is less than an insertion height of the trailing edge of the intervertebral prosthetic disc. As such, the intervertebral prosthetic disc can be held in a wedge shape, or ramp shape, during insertion. In a particular embodiment, holding the intervertebral prosthetic disc in a wedge shape can aid in distracting the intervertebral disc space and spreading the vertebrae apart. Further, an entrance gap of the intervertebral disc space may be smaller than the total height of the intervertebral prosthetic disc.

### Description of a Second Embodiment

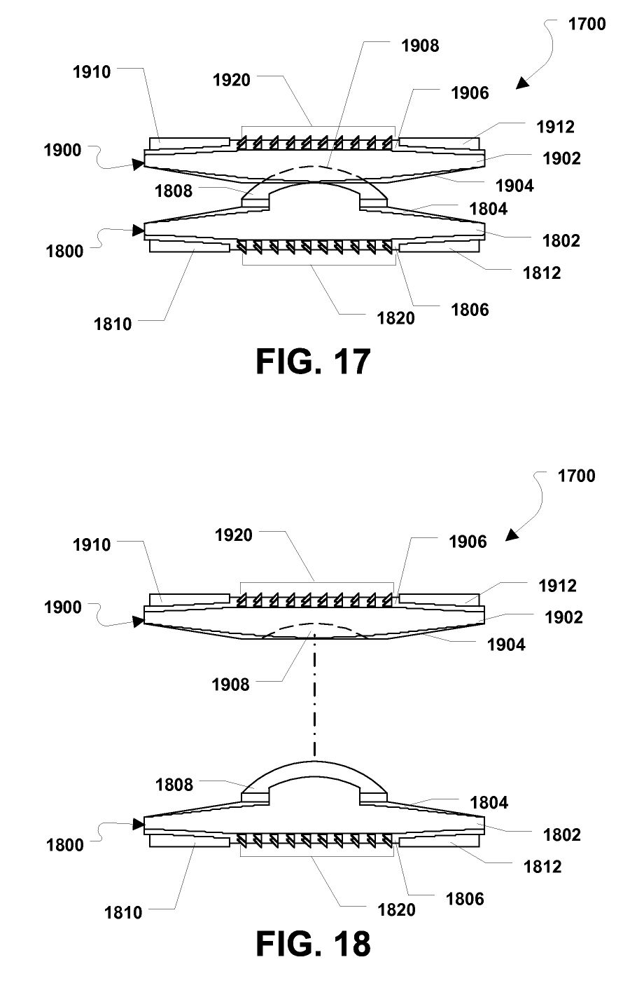

Referring to FIG. 17 through 22 a second embodiment of an intervertebral prosthetic disc is shown and is generally designated 1700. As illustrated, the intervertebral prosthetic disc 1700 includes an inferior articular half 1800 and a superior articular half 1900. In a particular embodiment, the articular halves 1800, 1900 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the articular halves 1800, 1900 can be made from any other biocompatible materials.

In a particular embodiment, the inferior articular half 1800 includes an inferior support plate 1802 that has an inferior articular surface 1804 and an inferior bearing surface 1806. In a particular embodiment, the inferior articular surface 1804 and the inferior bearing surface 1806 are generally rounded. In a particular embodiment, after installation the inferior bearing surface 1806 can be in direct contact with vertebral bone, e.g., cortical bone and cancellous bone. Further, the inferior bearing surface 1806 can be coated with a bone-growth promoting substance, e.g., a hydroxyapatite coating formed of calcium phosphate. Additionally, the inferior bearing surface 1806 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 17 through FIG. 21, a projection 1808 extends from the inferior articular surface 1804 of the inferior support plate 1802. In a particular embodiment, the projection 1808 has a hemi-spherical shape. Alternatively, the projection 1808 can have an elliptical shape, a cylindrical shape, or other arcuate shape.

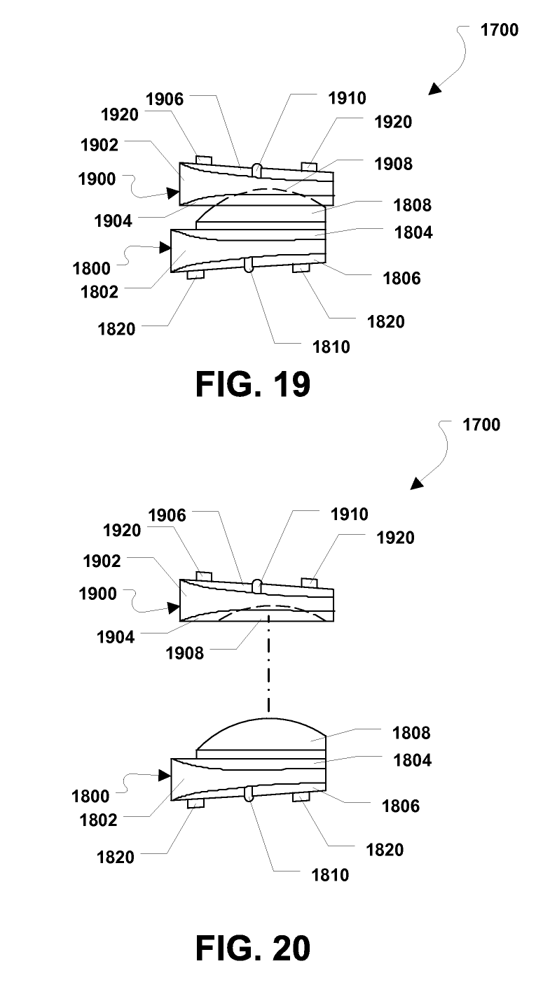

As further illustrated in FIG. 17 through 22, the inferior articular half 1800 includes an inferior rib 1810 that extend substantially perpendicularly from the inferior bearing surface 1806. In a particular embodiment, as shown in FIG. 22, the inferior rib 1810 extends along a longitudinal axis 1814 defined by the inferior articular half 1800. As shown, the inferior rib 1810 can extend along the longitudinal axis 1814 along the entire length of the inferior bearing surface 1806 parallel to the longitudinal axis 1814 and perpendicular to a lateral axis 1816 that is defined by the inferior articular half 1800. In particular embodiment, the inferior rib 1810 is sized and shaped to engage a first and second slot formed within a cortical rim of an inferior vertebra. Further, as shown in FIG. 19 and FIG. 20, the inferior rib 1810 can rounded, e.g., the inferior rib 1810 can have a semi-circular cross-section or an elliptical cross-section.

FIG. 17 through FIG. 22 also show that the inferior articular half 1800 includes a plurality of inferior teeth 1820 that extend from the inferior bearing surface 1806. As shown, a first row of inferior teeth 1820 and a second row of inferior teeth 1820 extend from the inferior bearing surface 1806 between the inferior rib 1810 and an anterior side of the inferior articular half 1800. Moreover, a third row of inferior teeth 1820 and a fourth row of inferior teeth 1820 extend from the inferior bearing surface 1806 between the inferior rib 1810 and a proximal side of the inferior articular half 1800. Further, in a particular embodiment, the inferior teeth 1820 are generally saw-tooth, or triangle, shaped. Also, the inferior teeth 1820 can be designed to engage cancellous bone of an inferior vertebra. Additionally, the inferior teeth 1820 can prevent the inferior articular half 1800 from moving with respect to an inferior vertebra after the intervertebral prosthetic disc 1700 is installed within an intervertebral space between an inferior vertebra and a superior vertebra.

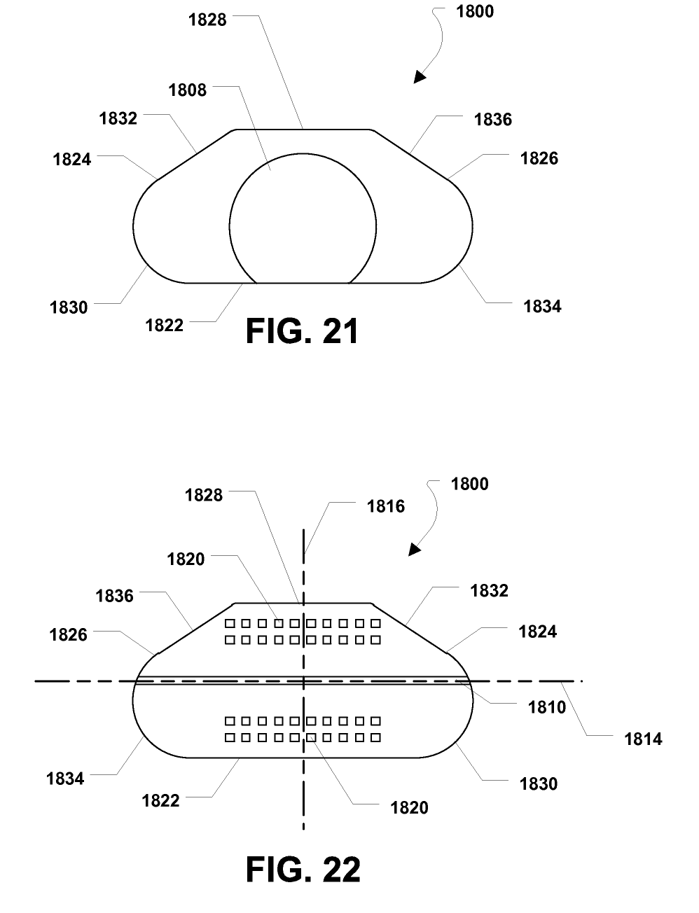

As illustrated in FIG. 21 and FIG. 22, the inferior articular half 1800 can be generally shaped to match the general shape of the vertebral body of a vertebra, e.g., the vertebral body 304 of the inferior vertebra 302 shown in FIG. 3. For example, the inferior articular half 1800 can have a general trapezoid shape and the inferior articular half 1800 can include a posterior side 1822. A first lateral side 1824 and a second lateral side 1826 can extend from the posterior side 1822 to an anterior side 1828. In a particular embodiment, the first lateral side 1824 includes a curved portion 1830 and a straight portion 1832 that extends at an angle toward the anterior side 1828. Further, the second lateral side 1826 can also include a curved portion 1834 and a straight portion 1836 that extends at an angle toward the anterior side 1828.

As shown in FIG. 21 and FIG. 22, the anterior side 1828 of the inferior articular half 1800 can be relatively shorter than the posterior side 1822 of the inferior articular half 1800. Further, in a particular embodiment, the anterior side 1828 is substantially parallel to the posterior side 1822. As indicated in FIG. 8, the projection 1808 is situated, or otherwise formed, on the inferior articular surface 1804 such that the perimeter of the projection 1808 is partially truncated by the posterior side 1822 of the inferior articular half 1800. As such, the projection 1808 is shifted in a posterior direction relative to the location of the projection 508 within the first embodiment of the intervertebral prosthetic disc 400. In a particular embodiment, the projection 1808 is shifted in the posterior direction relative to the location of the projection 508 of the first embodiment of the intervertebral prosthetic disc 400 in a range of one millimeter to six millimeters (1-6 mm). Moving the projection 1808 can account for variations in spinal alignment from patient to patient.

FIG. 17 through 22 illustrates that the superior articular half 1900 of the intervertebral prosthetic disc 1700 can include a superior support plate 1902 that has a superior articular surface 1904 and a superior bearing surface 1906. In a particular embodiment, the superior articular surface 1904 and the superior bearing surface 1906 are generally rounded. In a particular embodiment, after installation the superior bearing surface 1906 can be in direct contact with vertebral bone, e.g., cortical bone and cancellous bone. Further, the superior bearing surface 1906 can be coated with a bone-growth promoting substance, e.g., a hydroxyapatite coating formed of calcium phosphate. Additionally, the superior bearing surface 1906 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 17 through FIG. 20, a depression 1908 can extend into the superior articular surface 1904 of the superior support plate 1902. In a particular embodiment, the depression 1908 is sized and shaped to receive the projection 1808 of the inferior articular half 1800. For example, the depression 1908 can have a hemi-spherical shape. Alternatively, the depression 1908 can have an elliptical shape, a cylindrical shape, or other arcuate shape.

As further illustrated in FIG. 17 through 20, the superior articular half 1900 includes a superior rib 1910 that extend substantially perpendicularly from the superior bearing surface 1906. In a particular embodiment, the superior rib 1910 of the superior articular half 1900 can be arranged in a manner similar to the inferior rib 1810 of the inferior articular half 1800, as shown in FIG. 22. In another particular embodiment, the superior rib 1910 is sized and shaped to engage a first and second slot that can be established within a cortical rim of a superior vertebra.

FIG. 17 through FIG. 20 also show that the superior articular half 1900 includes a plurality of superior teeth 1920 that extend from the superior bearing surface 1906. In a particular embodiment, the superior teeth 1920 can be arranged in a pattern that is substantially the same as the plurality of inferior teeth 1820 that extend from the inferior bearing surface 1806 of the inferior articular half 1800. As shown, in a particular embodiment, the superior teeth 1920 are generally saw-tooth, or triangle, shaped. Further, the superior teeth 1920 are designed to engage cancellous bone of a superior vertebra. Additionally, the superior teeth 1920 can prevent the superior articular half 1900 from moving with respect to a superior vertebra after the intervertebral prosthetic disc 1700 is installed within the intervertebral space between an inferior vertebra and the superior vertebra.

In a particular embodiment, the superior articular half 1900 can be shaped to match the shape of the inferior articular half 1800, shown in FIG. 21 and FIG. 22. Further, the superior articular half 1900 can be shaped to match the general shape of the vertebral body of a vertebra, e.g., the vertebral body 304 of the superior vertebra 300 shown in FIG. 3.

In a particular embodiment, the overall height of the intervertebral prosthetic device 1700 can be in a range from six millimeters to twenty-two millimeters (6-22 mm). Further, the installed height of the intervertebral prosthetic device 1700 can be in a range from four millimeters to sixteen millimeters (4-16 mm). In a particular embodiment, the installed height can be substantially equivalent to the distance between an inferior vertebra and a superior vertebra when the intervertebral prosthetic device 1700 is installed there between.

In a particular embodiment, the length of the intervertebral prosthetic device 1700, e.g., along a longitudinal axis, can be in a range from thirty-three millimeters to fifty millimeters (33-50 mm). Additionally, the width of the intervertebral prosthetic device 1700, e.g., along a lateral axis, can be in a range from eighteen millimeters to twenty-nine millimeters (18-29 mm). Moreover, in a particular embodiment, each rib 1810, 1910 can have a height in a range from one millimeter to six millimeters (1-6 mm). In a particular embodiment, the height of each rib 1810, 1910 is measured at a location of each rib 1810, 1910 nearest to the center of each half 1800, 1900 of the intervertebral prosthetic device 1700.

In a particular embodiment, the ribs 1810, 1910 can be considered “low profile”. Further, intervertebral prosthetic disc 1700 can be considered to be “low profile.” The low profile of the ribs 1810, 1910 and the intervertebral prosthetic device 1700 can allow the intervertebral prosthetic device 1700 to be implanted into an intervertebral space between an inferior vertebra and a superior vertebra laterally through a patient's psoas muscle. Accordingly, the risk of damage to a patient's spinal cord or sympathetic chain can be substantially minimized. In alternative embodiments, all of the superior and inferior teeth 1820, 1920 can be oriented to engage in a direction substantially opposite the direction of insertion of the prosthetic disc into the intervertebral space.

Further, the intervertebral prosthetic disc 1700 can have a general “bullet” shape as shown in the posterior plan view, described herein. The bullet shape of the intervertebral prosthetic disc 1700 provided by the rounded bearing surfaces 1804, 1904 can further allow the intervertebral prosthetic disc 1700 to be inserted through the patient's psoas muscle while minimizing risk to the patient's spinal cord and sympathetic chain.

### Description of a Third Embodiment

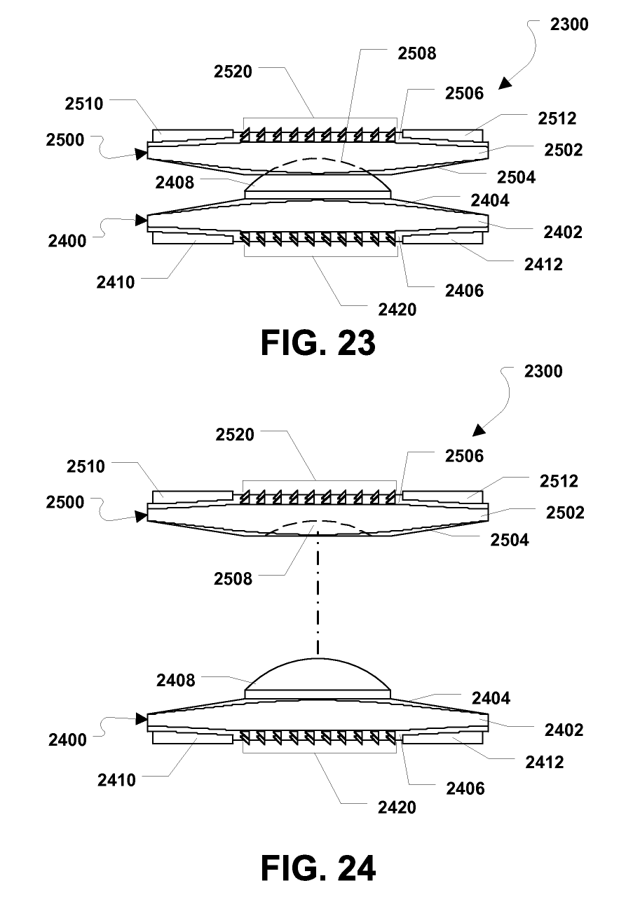

Referring to FIG. 23 through 27 a first embodiment of an intervertebral prosthetic disc is shown and is generally designated 2300. As illustrated, the intervertebral prosthetic disc 2300 includes an inferior articular half 2400 and a superior articular half 2500. In one embodiment, the articular halves 2400, 2500 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the articular halves 2400, 2500 can be made from any other biocompatible materials.

In a particular embodiment, the inferior articular half 2400 includes an inferior support plate 2402 that has an inferior articular surface 2404 and an inferior bearing surface 2406. In a particular embodiment, the inferior articular surface 2404 and the inferior bearing surface 2406 are generally rounded. In a particular embodiment, after installation the inferior bearing surface 2406 can be in direct contact with vertebral bone, e.g., cortical bone and cancellous bone. Further, the inferior bearing surface 2406 can be coated with a bone-growth promoting substance, e.g., a hydroxyapatite coating formed of calcium phosphate. Additionally, the inferior bearing surface 2406 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 23 through FIG. 27, a projection 2408 extends from the inferior articular surface 2404 of the inferior support plate 2402. In a particular embodiment, the projection 2408 has a hemi-spherical shape. Alternatively, the projection 2408 can have an elliptical shape, a cylindrical shape, or other arcuate shape.

As further illustrated in FIG. 23 through 27, the inferior articular half 2400 includes a first inferior rib 2410 and a second inferior rib 2412 that extend substantially perpendicularly from the inferior bearing surface 2406. In a particular embodiment, the first inferior rib 2410 and the second inferior rib 2412 can be arranged as described above in conjunction with the first embodiment of the intervertebral prosthetic disc 400. Additionally, in particular embodiment, the first inferior rib 2410 and the second inferior rib 2412 are sized and shaped to engage a first and second slot that can be established within a cortical rim of an inferior vertebra.

FIG. 23 through FIG. 27 also show that the inferior articular half 2400 includes a plurality of inferior teeth 2420 that extend from the inferior bearing surface 2406. In a particular embodiment, the plurality of inferior teeth 2420 are arranged in a pattern similar to one of the patterns illustrated in conjunction with the first embodiment of the intervertebral prosthetic disc 400 and the second embodiment of the intervertebral prosthetic disc 1600. As shown, in a particular embodiment, the inferior teeth 2420 are generally saw-tooth, or triangle, shaped. Further, the inferior teeth 2420 are designed to engage cancellous bone of an inferior vertebra. Additionally, the inferior teeth 2420 can prevent the inferior articular half 2400 from moving with respect to an inferior vertebra after the intervertebral prosthetic disc is installed within an intervertebral space between an inferior vertebra and a superior vertebra.

As illustrated in FIG. 26, the inferior articular half 2400 can be generally shaped to match the general shape of the vertebral body of a vertebra, e.g., the vertebral body 304 of the inferior vertebra 302 shown in FIG. 3. For example, the inferior articular half 2400 can have a general trapezoid shape and the inferior articular half 2400 can include a posterior side 2422. A first lateral side 2424 and a second lateral side 2426 can extend from the posterior side 2422 to an anterior side 2428. In a particular embodiment, the first lateral side 2424 includes a curved portion 2430 and a straight portion 2432 that extends at an angle toward the anterior side 2428. Further, the second lateral side 2426 can also include a curved portion 2434 and a straight portion 2436 that extends at an angle toward the anterior side 2428.

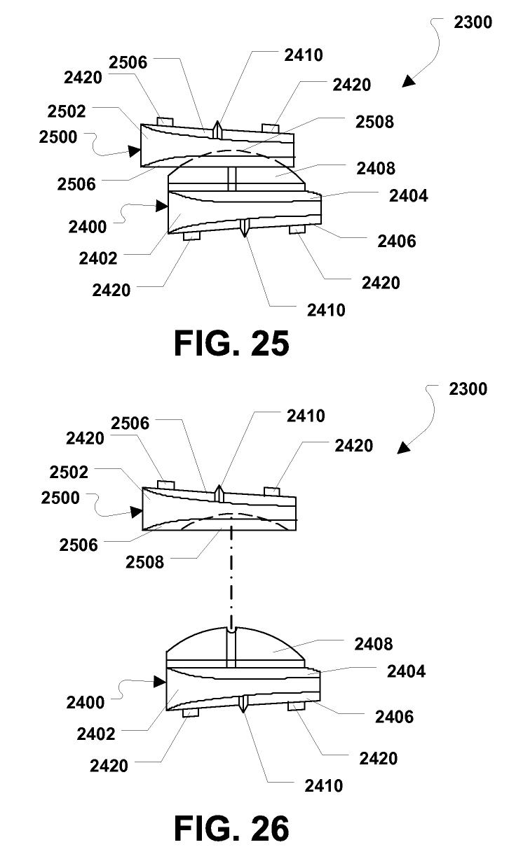

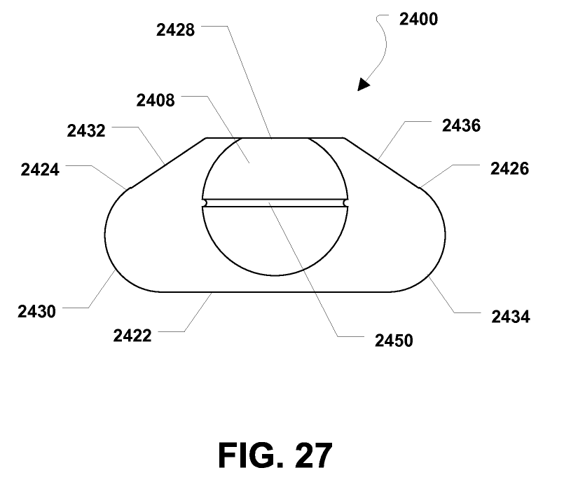

As shown in FIG. 27, the anterior side 2428 of the inferior articular half 2400 can be relatively shorter than the posterior side 2422 of the inferior articular half 2400. Further, in a particular embodiment, the anterior side 2428 is substantially parallel to the posterior side 2422. As shown in FIG. 25 through 27, the projection can be formed with a groove 2450 along a surface of the projection 2408.

As indicated in FIG. 27, the projection 2408 is situated, or otherwise formed, on the inferior articular surface 2404 such that the perimeter of the projection 2408 is partially truncated by the anterior side 2428 of the inferior articular half 2400. As such, the projection 2408 is shifted in an anterior direction relative to the location of the projection 508 within the first embodiment of the intervertebral prosthetic disc 400. In a particular embodiment, the projection 2408 is shifted in the anterior direction relative to the location of the projection 508 of the first embodiment of the intervertebral prosthetic disc 400 in a range of one millimeter to six millimeters (1-6 mm). Moving the projection 2408 can account for variations in spinal alignment from patient to patient.

In a particular embodiment, the superior articular half 2500 includes a superior support plate 2502 that has a superior articular surface 2504 and a superior bearing surface 2506. In a particular embodiment, the superior articular surface 2504 and the superior bearing surface 2506 are generally rounded. In a particular embodiment, after installation the superior bearing surface 2506 can be in direct contact with vertebral bone, e.g., cortical bone and cancellous bone. Further, the superior bearing surface 2506 can be coated with a bone-growth promoting substance, e.g., a hydroxyapatite coating formed of calcium phosphate. Additionally, the superior bearing surface 2506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 23 through FIG. 26, a depression 2508 extends into the superior articular surface 2504 of the superior support plate 2502. In a particular embodiment, the depression 2508 is sized and shaped to receive the projection 2408 of the inferior articular half 2400. For example, the depression 2508 can have a hemi-spherical shape. Alternatively, the depression 2508 can have an elliptical shape, a cylindrical shape, or other arcuate shape.

As further illustrated in FIG. 23 through 26, the superior articular half 2500 includes a first superior rib 2510 and a second superior rib 2512 that extend substantially perpendicularly from the superior bearing surface 2506. In a particular embodiment, the first superior rib 2510 and the second superior rib 2512 of the superior articular half 2500 are arranged in a manner similar to the first inferior rib 2410 and the second inferior rib 2412 of the inferior articular half 2400, as shown in FIG. 9. In another particular embodiment, the first superior rib 2510 and the second superior rib 2512 are sized and shaped to engage a first and second slot that can be formed within a cortical rim of a superior vertebra.

FIG. 23 through FIG. 26 also show that the superior articular half 2500 includes a plurality of superior teeth 2520 that extend from the superior bearing surface 2506. As shown, in a particular embodiment, the superior teeth 2520 are generally saw-tooth, or triangle, shaped. In a particular embodiment, the superior teeth 2520 are arranged in a pattern that is substantially similar to the teeth 2430 on the inferior articular half 2400. Further, the superior teeth 2520 are designed to engage cancellous bone of a superior vertebra. Additionally, the superior teeth 2520 can prevent the superior articular half 2500 from moving with respect to a superior vertebra after the intervertebral prosthetic disc 2300 is installed within an intervertebral space between an inferior vertebra and the superior vertebra.

In a particular embodiment, the superior articular half 2500 can be shaped to match the shape of the inferior articular half 2400, shown in FIG. 26. Further, the superior articular half 2500 can be shaped to match the general shape of the vertebral body of a vertebra, e.g., the vertebral body 304 of the superior vertebra 302 shown in FIG. 3.

In a particular embodiment, the overall height of the intervertebral prosthetic device 2300 can be in a range from six millimeters to twenty-two millimeters (6-23 mm). Further, the installed height of the intervertebral prosthetic device 2300 can be in a range from four millimeters to sixteen millimeters (4-16 mm). In a particular embodiment, the installed height can be substantially equivalent to the distance between an inferior vertebra and a superior vertebra when the intervertebral prosthetic device 2300 is installed there between.

In a particular embodiment, the length of the intervertebral prosthetic device 2300, e.g., along a longitudinal axis, can be in a range from thirty-three millimeters to fifty millimeters (33-50 mm). Additionally, the width of the intervertebral prosthetic device 2300, e.g., along a lateral axis, can be in a range from eighteen millimeters to twenty-nine millimeters (18-29 mm). Moreover, in a particular embodiment, each rib 2410, 2412, 2510, 2512 can have a height in a range from one millimeter to six millimeters (1-6 mm). In a particular embodiment, the height of each rib 2410, 2412, 2510, 2512 is measured at a location of each rib 2410, 2412, 2510, 2512 nearest to the center of each half 2400, 2500 of the intervertebral prosthetic device 2300.

In a particular embodiment, the ribs 2410, 2412, 2510, 2512 can be considered “low profile”. Further, intervertebral prosthetic disc 2300 can be considered to be “low profile.” The low profile of the ribs 2410, 2412, 2510, 2512 and the intervertebral prosthetic device 2300 can allow the intervertebral prosthetic device 2300 to be implanted into an intervertebral space between an inferior vertebra and a superior vertebra laterally through a patient's psoas muscle. Accordingly, the risk of damage to a patient's spinal cord or sympathetic chain can be substantially minimized. In alternative embodiments, all of the superior and inferior teeth 2420, 2520 can be oriented to engage in a direction substantially opposite the direction of insertion of the prosthetic disc into the intervertebral space.

Further, the intervertebral prosthetic disc 2300 can have a general “bullet” shape as shown in the posterior plan view, described herein. The bullet shape of the intervertebral prosthetic disc 2300 provided by the rounded bearing surfaces 2404, 2504 can further allow the intervertebral prosthetic disc 2300 to be inserted through the patient's psoas muscle while minimizing risk to the patient's spinal cord and sympathetic chain.

### Description of a Fourth Embodiment

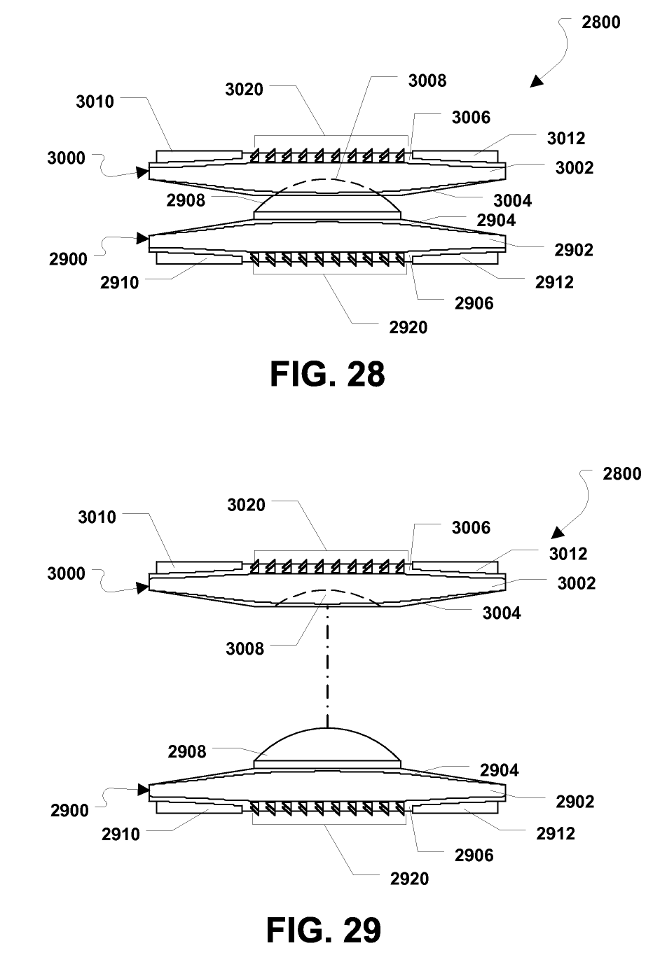

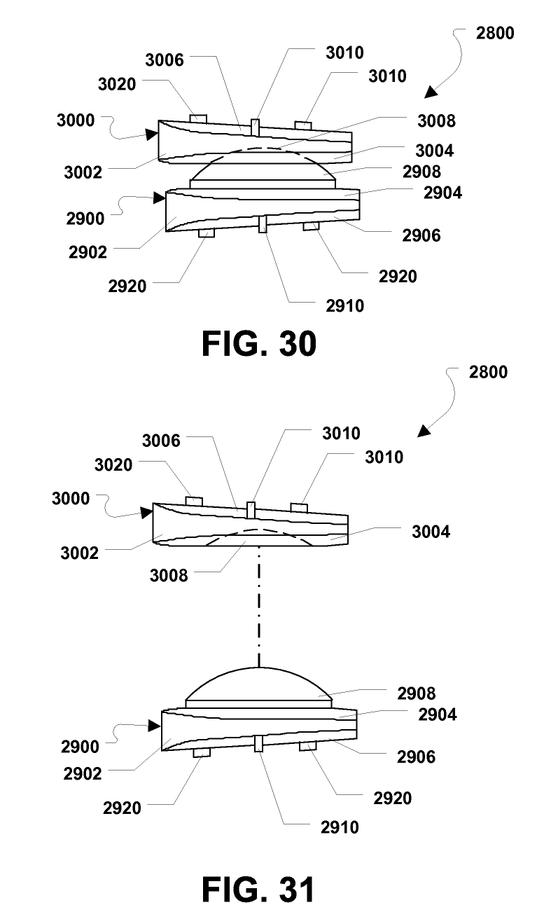

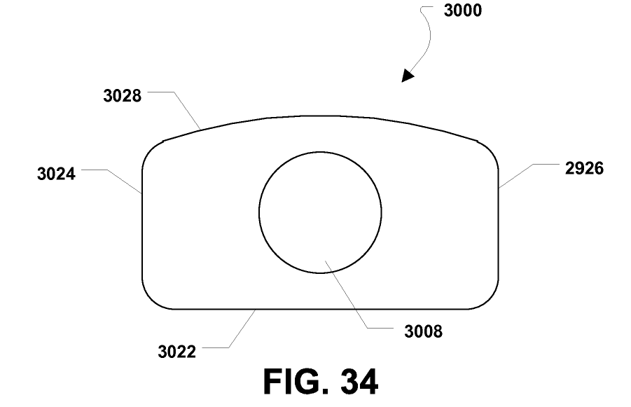

Referring to FIG. 28 through FIG. 34 a fourth embodiment of an intervertebral prosthetic disc is shown and is generally designated 2800. As illustrated, the intervertebral prosthetic disc 2800 includes an inferior articular half 2900 and a superior articular half 3000. In one embodiment, the articular halves 2900, 3000 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the articular halves 2900, 3000 can be made from any other biocompatible materials.

In a particular embodiment, the inferior articular half 2900 includes an inferior support plate 2902 that has an inferior articular surface 2904 and an inferior bearing surface 2906. In a particular embodiment, the inferior articular surface 2904 and the inferior bearing surface 2906 are generally rounded. In a particular embodiment, after installation the inferior bearing surface 2906 can be in direct contact with vertebral bone, e.g., cortical bone and cancellous bone. Further, the inferior bearing surface 2906 can be coated with a bone-growth promoting substance, e.g., a hydroxyapatite coating formed of calcium phosphate. Additionally, the inferior bearing surface 2906 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 28 through FIG. 34, a projection 2908 extends from the inferior articular surface 2904 of the inferior support plate 2902. In a particular embodiment, the projection 2908 has a hemi-spherical shape. Alternatively, the projection 2908 can have an elliptical shape, a cylindrical shape, or other arcuate shape.

As further illustrated in FIG. 28 through 34, the inferior articular half 2900 includes a first inferior rib 2910 and a second inferior rib 2912 that extend substantially perpendicularly from the inferior bearing surface 2906. In a particular embodiment, as shown in FIG. 33, the first inferior rib 2910 and the second inferior rib 2912 extend along a longitudinal axis 2914 defined by the inferior articular half 2900. As shown, the first inferior rib 2910 and the second inferior rib 2912 can extend along the longitudinal axis 2914 from a perimeter of the inferior articular half 2900 toward a lateral axis 2916 that is defined by the inferior articular half 2900. In particular embodiment, the first inferior rib 2910 and the second inferior rib 2912 are sized and shaped to engage a first and second slot that can be established within a cortical rim of an inferior vertebra.

FIG. 28 through FIG. 34 also show that the inferior articular half 2900 includes a plurality of inferior teeth 2920 that extend from the inferior bearing surface 2906. As shown, in a particular embodiment, the inferior teeth 2920 are generally saw-tooth, or triangle, shaped. Further, the inferior teeth 2920 are designed to engage cancellous bone of an inferior vertebra. Additionally, the inferior teeth 2920 can prevent the inferior articular half 2900 from moving with respect to an inferior vertebra after the intervertebral prosthetic disc 2800 is installed within an intervertebral space between an inferior vertebra and a superior vertebra.

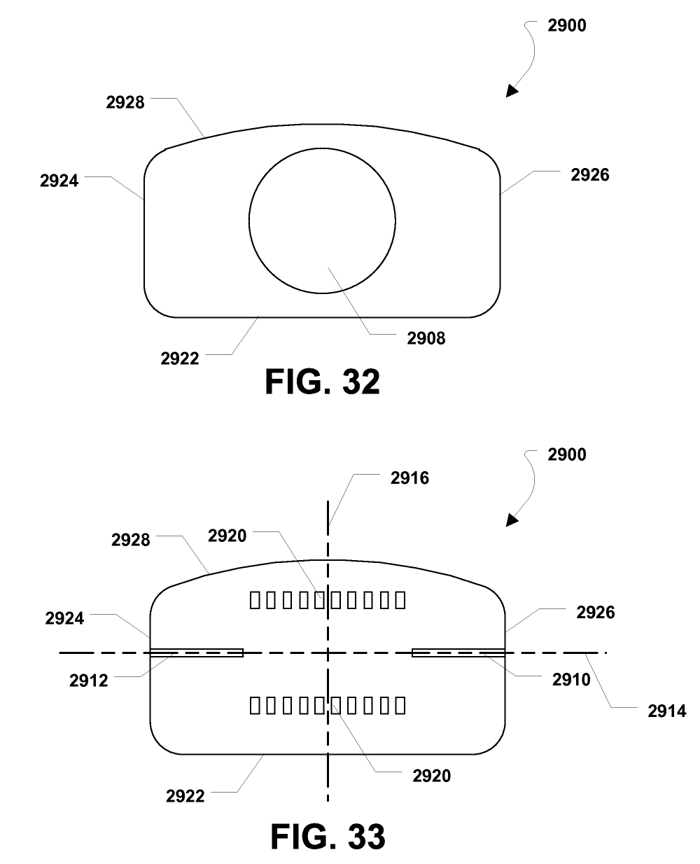

As illustrated in FIG. 32 and FIG. 33, the inferior articular half 2900 can be generally rectangular in shape. For example, the inferior articular half 2900 can have a substantially straight posterior side 2922. A first straight lateral side 2924 and a second substantially straight lateral side 2926 can extend substantially perpendicular from the posterior side 2922 to an anterior side 2928. In a particular embodiment, the anterior side 2928 can curve outward such that the inferior articular half 2900 is wider through the middle than along the lateral sides 2924, 2926. Further, in a particular embodiment, the lateral sides 2924, 2926 are substantially the same length.

As shown in FIG. 28 through 34, the superior articular half 3000 includes a superior support plate 3002 that has a superior articular surface 3004 and a superior bearing surface 3006. In a particular embodiment, the superior articular surface 3004 and the superior bearing surface 3006 are generally rounded. In a particular embodiment, after installation the superior bearing surface 3006 can be in direct contact with vertebral bone, e.g., cortical bone and cancellous bone. Further, the superior bearing surface 3006 can be coated with a bone-growth promoting substance, e.g., a hydroxyapatite coating formed of calcium phosphate. Additionally, the superior bearing surface 3006 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray; e.g., titanium plasma spray (TPS); laser blasting; or any other similar process or method.

As illustrated in FIG. 28 through FIG. 34, a depression 3008 extends into the superior articular surface 3004 of the superior support plate 3002. In a particular embodiment, the depression 3008 is sized and shaped to receive the projection 2908 of the inferior articular half 2900. For example, the depression 3008 can have a hemi-spherical shape. Alternatively, the depression 3008 can have an elliptical shape, a cylindrical shape, or other arcuate shape.

As further illustrated in FIG. 28 through 34, the superior articular half 3000 includes a first superior rib 3010 and a second superior rib 3012 that extend substantially perpendicularly from the superior bearing surface 3006. In a particular embodiment, the first superior rib 3010 and the second superior rib 3012 of the superior articular half 3000 are arranged in a manner similar to the first inferior rib 2910 and the second inferior rib 2912 of the inferior articular half 2900, as shown in FIG. 33. In another particular embodiment, the first superior rib 3010 and the second superior rib 3012 are sized and shaped to engage a first and second slot that can be established within the cortical rim of a superior vertebra.

FIG. 28 through FIG. 34 also show that the superior articular half 3000 includes a plurality of superior teeth 3020 that extend from the superior bearing surface 3006. As shown, in a particular embodiment, the superior teeth 3020 are generally saw-tooth, or triangle, shaped. Also, in a particular embodiment, the superior teeth 3020 of the superior articular half 3000 are arranged in a manner similar to the inferior teeth 2920 of the inferior articular half 2900, as shown in FIG. 33. Further, the superior teeth 3020 are designed to engage cancellous bone of a superior vertebra. Additionally, the superior teeth 3020 can prevent the superior articular half 3000 from moving with respect to a superior vertebra after the intervertebral prosthetic disc 2800 is installed within an intervertebral space between the superior vertebra and the superior vertebra.

In a particular embodiment, the superior articular half 3000 can be shaped to match the shape of the inferior articular half 2900, shown in FIG. 32 and FIG. 33. Further, the superior articular half 3000 can be generally rectangular in shape. For example, the superior articular half 3000 can have a substantially straight posterior side 3022. A first straight lateral side 3024 and a second substantially straight lateral side 3026 can extend substantially perpendicular from the posterior side 3022 to an anterior side 3028. In a particular embodiment, the anterior side 3028 can curve outward such that the superior articular half 3000 is wider through the middle than along the lateral sides 3024, 3026. Further, in a particular embodiment, the lateral sides 3024, 3026 are substantially the same length.

In a particular embodiment, the overall height of the intervertebral prosthetic device 2800 can be in a range from six millimeters to twenty-two millimeters (6-22 mm). Further, the installed height of the intervertebral prosthetic device 2800 can be in a range from four millimeters to sixteen millimeters (4-16 mm). In a particular embodiment, the installed height can be substantially equivalent to the distance between an inferior vertebra and a superior vertebra when the intervertebral prosthetic device 2800 is installed there between.

In a particular embodiment, the length of the intervertebral prosthetic device 2800, e.g. along a longitudinal axis, can be in a range from thirty-three millimeters to fifty millimeters (33-50 mm). Additionally, the width of the intervertebral prosthetic device 2800, e.g., along a lateral axis, can be in a range from eighteen millimeters to twenty-nine millimeters (18-29 mm). Moreover, in a particular embodiment, each rib 2910, 2912, 3010, 3012 can have a height in a range from one millimeter to six millimeters (1-6 mm). In a particular embodiment, the height of each rib 2910, 2912, 3010, 3012 is measured at a location of each rib 2910, 2912, 3010, 3012 nearest to the center of each half 2900, 3000 of the intervertebral prosthetic device 2800.

In a particular embodiment, the ribs 2910, 2912, 3010, 3012 can be considered “low profile”. Further, intervertebral prosthetic disc 2800 can be considered to be “low profile.” The low profile of the ribs 2910, 2912, 3010, 3012 and the intervertebral prosthetic device 2800 can allow the intervertebral prosthetic device 2800 to be implanted into an intervertebral space between an inferior vertebra and a superior vertebra laterally through a patient's psoas muscle. Accordingly, the risk of damage to a patient's spinal cord or sympathetic chain can be substantially minimized. In alternative embodiments, all of the superior and inferior teeth 2920, 3020 can be oriented to engage in a direction substantially opposite the direction of insertion of the prosthetic disc into the intervertebral space.

Further, the intervertebral prosthetic disc 2800 can have a general “bullet” shape shown in the posterior plan view, described herein. The bullet shape of the intervertebral prosthetic disc 2800 provided by the rounded bearing surfaces 2904, 3004 can further allow the intervertebral prosthetic disc 2800 to be inserted through the patient's psoas muscle while minimizing risk to the patient's spinal cord and sympathetic chain.

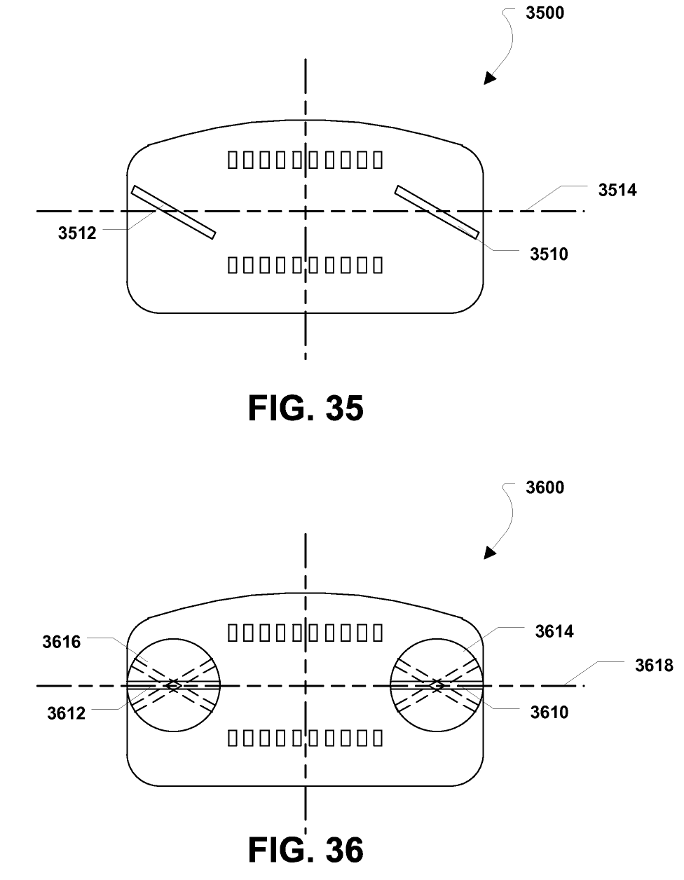

FIG. 35 illustrates another inferior articular half, designated 3500. The articular half shown in FIG. 35 includes a first inferior rib 3510 and a second inferior rib 3512 that extend substantially perpendicularly from the inferior articular half 3500. In a particular embodiment, the first inferior rib 3510 and the second inferior rib 3512 are angled with respect to a longitudinal axis 3514 that is defined by the inferior articular half 3500. Further, the first inferior rib 3510 and the second inferior rib 3512 are substantially parallel to each other. In particular embodiment, the first inferior rib 3510 and the second inferior rib 3512 are sized and shaped to engage a first and second slot that can be established within a cortical rim of an inferior vertebra. In an alternative embodiment, the inferior articular half 3500 may include a single inferior rib that is angled with respect to the longitudinal axis 3514. In such, a case, the single inferior rib can extend along the entire length of the inferior articular half 3500.

In a particular embodiment, the inferior articular half 3500 can cooperate with a superior articular half (not shown in FIG. 35) to provide relative motion between a superior vertebra and an inferior vertebra. The superior articular half can includes superior ribs that are substantially similar to the inferior ribs 3510, 3512 of the inferior articular half 3500. Further, the angled ribs can facilitate another surgical approach for installing an intervertebral prosthetic disc, e.g., an anterior approach, a posterior approach, or any other surgical approach.

FIG. 36 illustrates yet another inferior articular half, designated 3600. The articular half shown in FIG. 36 includes a first inferior rib 3610 and a second inferior rib 3612 that extend substantially perpendicularly from the inferior articular half 3600. In a particular embodiment, the first inferior rib 3610 is disposed on, or attached to, a first rotatable disc 3614 that is incorporated into the inferior articular half 3600. Also, the second inferior rib 3612 is disposed on, or attached to, a second rotatable disc 3616 that is incorporated into the inferior articular half 3600. As such, the inferior ribs 3610, 3612 can be rotated with respect to a longitudinal axis 3618 that is defined by the inferior articular half 3500. In particular embodiment, the first inferior rib 3610 and the second inferior rib 3612 are sized and shaped to engage a first and second slot that can be established within a cortical rim of an inferior vertebra.

In a particular embodiment, the inferior articular half 3600 can cooperate with a superior articular half (not shown in FIG. 36) to provide relative motion between a superior vertebra and an inferior vertebra. The superior articular half can include superior ribs that are substantially similar to the inferior ribs 3610, 3612 of the inferior articular half 3600. Further, the rotatable ribs can facilitate another surgical approach for installing an intervertebral prosthetic disc, e.g., an anterior approach, a posterior approach, or any other surgical approach.

### CONCLUSION

With the configuration of structure described above, the intervertebral prosthetic disc according to one or more of the embodiments provides a device that may be implanted to replace a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

During surgery, a surgeon may plan to implant a fusion device. However, as the surgery progresses and the surgeon is able to more accurately determine the condition of the end plate of the inferior vertebra and the condition of the end plate of the superior vertebra, the surgeon may choose to implant the intervertebral prosthetic disc according to one of the embodiments described herein in lieu of implanting a fusion device. As such, the patient may be given a chance to recover from the surgery with greater mobility that the mobility provided by a fusion device.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc configured to be installed within an intervertebral space established between an inferior vertebra and a superior vertebra, the intervertebral prosthetic disc comprising:
an inferior articular half configured to engage the inferior vertebra;
a superior articular half configured to engage the superior vertebra;
wherein the inferior articular half is configured to cooperate with the superior articular half to allow relative angular motion between the inferior vertebra and the superior vertebra when installed; and
wherein the intervertebral prosthetic device is sized and shaped to pass through a psoas muscle without injuring a spinal cord or a sympathetic chain.

2. The intervertebral prosthetic disc of claim 1, wherein the intervertebral prosthetic disc is sized and shaped to pass laterally through the psoas muscle.

3. (canceled)

4. The intervertebral prosthetic disc of claim 2, wherein the anatomic safe zone is less than or equal to three centimeters (3.0 cm) wide.

5. (canceled)

6. The intervertebral prosthetic disc of claim 2, wherein the intervertebral prosthetic disc is sized and shaped to pass through an installation device installed through the anatomic safe zone within the psoas muscle.

7.-9. (canceled)

10. The intervertebral prosthetic disc of claim 52, wherein the overall height of the intervertebral prosthetic disc is less than or equal to twenty-two millimeters (22 mm).

11. The intervertebral prosthetic disc of claim 10, wherein an overall height of the intervertebral prosthetic disc is greater or equal to six millimeters (6 mm).

12. The intervertebral prosthetic disc of claim 52, wherein the width of the intervertebral prosthetic disc is less than or equal to twenty-nine millimeters (29 mm).

13. The intervertebral prosthetic disc of claim 12, wherein a width of the intervertebral prosthetic disc is greater or equal to eighteen millimeters (18 mm).

14. The intervertebral prosthetic disc of claim 52, wherein the length of the intervertebral prosthetic disc is less than or equal to fifty millimeters (50 mm).

15. The intervertebral prosthetic disc of claim 14, wherein a length of the intervertebral prosthetic disc is greater or equal to thirty-three millimeters (33 mm).

16. An intervertebral prosthetic disc configured to be installed within an intervertebral space established between an inferior vertebra and a superior vertebra, the intervertebral prosthetic disc comprising:
an inferior articular half, the inferior articular half including:
an inferior articular surface;
a projection extending from the inferior articular surface;
an inferior bearing surface; and
an inferior rib extending from the inferior bearing
surface and configured to engage a cortical rim of the inferior vertebra, wherein the inferior rib has a height that is less than or equal to six millimeters (6 mm).

17. The intervertebral prosthetic disc of claim 16, further comprising:
a superior articular half, the superior articular half including:
a superior articular surface;
a depression formed within the superior articular surface, wherein the depression is sized and shaped to movably engage the projection;
a superior bearing surface; and
a superior rib extending from the superior bearing surface and configured to engage a cortical rim of the superior vertebra, wherein the superior rib has a height that is less than or equal to six millimeters (6 mm).

18. The intervertebral prosthetic disc of claim 17, wherein the height of the inferior rib is greater than or equal to one millimeter (1 mm) and wherein the height of the superior rib is greater than or equal to one millimeter (1 mm).

19. The intervertebral prosthetic disc of claim 18, wherein the inferior rib is configured to engage a slot within the cortical rim of the inferior vertebra and wherein the superior rib is configured to engage a slot within the cortical rim of the superior vertebra.

20. The intervertebral prosthetic disc of claim 17, wherein the inferior rib, the superior rib, or a combination thereof is angled with respect to a longitudinal axis defined by the inferior articular half.

21. The intervertebral prosthetic disc of claim 17, wherein the inferior rib, the superior rib, or a combination thereof is rotatable with respect to a longitudinal axis defined by the inferior articular half.

22. (canceled)

23. (canceled)

24. The intervertebral prosthetic disc of claim 16, wherein the intervertebral prosthetic disc is generally bullet shaped, from a posterior plan view, to facilitate implantation through a psoas muscle.

25. The intervertebral prosthetic disc of claim 16, wherein the intervertebral prosthetic disc is generally trapezoidally shaped, from a superior plan view, to closely resemble a shape of a vertebral body.

26. The intervertebral prosthetic disc of claim 16, wherein the intervertebral prosthetic disc is generally rectangular, from a superior plan view.

27.-51. (canceled)

52. An intervertebral prosthetic disc configured to be installed within an intervertebral space established between an inferior vertebra and a superior vertebra, the intervertebral prosthetic disc comprising:
an inferior articular half configured to engage the inferior vertebra, the inferior articular half including a inferior tooth configured to engage cancellous bone of the inferior vertebra to prevent the inferior articular half from moving with respect to the inferior vertebra;
a superior articular half configured to engage the superior vertebra, the superior articular half including a superior tooth configured to engage cancellous bone of the superior vertebra to prevent the superior articular half from moving with respect to the superior vertebra;
wherein the inferior articular half is configured to cooperate with the superior articular half to allow relative angular motion between the inferior vertebra and the superior vertebra when installed; and
wherein the intervertebral prosthetic device is sized and shaped to pass through a psoas muscle without injuring a spinal cord or a sympathetic chain and each of the inferior and superior teeth are oriented to engage in a direction substantially opposite a direction of insertion.
